(19) United States
(12) Patent Application Publication
(10) Pub. No.: US 2022/0240935 A1
(43) Pub. Date: Aug. 4, 2022

(54) WOUND CLOSURE DEVICE WITH WIDE SPACED MICROSTRUCTURES

(71) Applicants: Cheuk Yin Paul Leung, Bellevue, WA (US); Ron Berenson, Mercer Island, WA (US)

(72) Inventors: Cheuk Yin Paul Leung, Bellevue, WA (US); Ron Berenson, Mercer Island, WA (US)

(21) Appl. No.: 17/584,612

(22) Filed: Jan. 26, 2022

Related U.S. Application Data
(60) Provisional application No. 63/143,215, filed on Jan. 29, 2021.

(51) Int. Cl. A61B 17/08

(52) U.S. Cl. CPC A61B 17/085; A61B 2017/086

(57) ABSTRACT

A wound closure device comprises a backing comprising a width configured to extend along at least a portion of a wound from a first end to a second end and a length configured to extend transversely across the wound, an adhesive layer coupled to the backing, a first micro-structure array attached to the backing proximate the first end, the first micro-structure array configured to extend transversely across the wound, a second micro-structure array attached to the backing proximate the second end, the second micro-structure array configured to extend transversely across the wound, and an elongate wound closure space between the first and second micro-structure arrays.

### CLAIM OF PRIORITY

This application claims priority to U.S. Provisional Application No. 63/143,215, filed on Jan. 29, 2021, which is herein incorporated by reference in its entirety.

### CROSS-REFERENCE TO RELATED PATENT DOCUMENTS

The present application is related to US Patent Publication Nos. 2015/0305739; and 2017/0333039; the entire contents of which are incorporated herein by reference.

### BACKGROUND

Existing devices, compositions, and methods for closing and treating a wound may range from simple over-the-counter products, such as dressings, wraps, bandages, adhesive bandages, butterfly strips, and surgical tape, to more specialized products, such as sutures and staples, depending on the type and severity of the wound, the skill of the caregiver, etc.

Although sutures and staples can be quite effective at closing wounds, proper application requires a trained specialist. Results are variable dependent on the skills of the specialist performing the closure procedure. Additionally, the application of sutures or staples is an invasive and painful procedure that frequently requires the use of an anesthetic. Furthermore, these procedures can leave unsightly scars, both from secondary insertion holes and from varying tensions applied to the laceration or surgical incision as a result of variations in suture or staple spacing and depth. Tension can lead to inflammation that causes scarring. Also, sutures and staples can increase risk of infection. Gaps in the wound between insertion sites of sutures and staples are portals for entry of bacteria and other infectious agents. Sutures and staples can enter the wound which can cause secondary tissue damage and lead to tracking of bacteria directly into the wound. For sutures, tension also can vary depending how tightly the suture is tied. Tied too tightly, tissue strangulation can occur leading to tissue necrosis, scarring, and risk of infection. Tied too loosely, the wound can open up risking infection. Moreover, these skin closure techniques can necessitate follow-up visits to a hospital or doctor's office for removal of the sutures or staples. This can be a problem not only for scheduled removal, but an even bigger issue if infection occurs since it requires removal of the sutures or sutures to reopen and clean the wound. Additionally, simply covering the wound with a bandage, such as an adhesive bandage, a butterfly closure strip, or surgical tape, is usually not sufficient to close more severe or deeper wounds, such as dermal wounds. This is because the adhesives used to attach devices such as adhesive bandages, butterfly closure strips, and surgical tape are not adequate to close these wounds without detaching or creep. Skin moisture can add to the problem by further reducing adherence of the adhesive-based bandage to the skin, which may lead to the premature release of the bandage from the skin and wound site before closure of the wound and proper healing.

For at least some of these reasons, improved wound closure devices are desirable. At least some of these issues will be addressed by the examples of wound closure devices described herein.

### OVERVIEW

The present inventors have recognized problems associated with wound closure devices. Sutures and staples have limited conformability and can cause tissue damage leading to infections and scarring. Bandages are generally conforming but do not have the strength to close most wounds. There is an unmet clinical need for a device that is both conforming and has the strength to close serious (e.g., deep dermal) wounds.

Many recent wound closure devices have incorporated micro-structures that facilitate gripping of tissue by the wound closure device through the use of micro-barbs, micro-staples or the like. These devices can be effective in closing wounds without the significant tissue injury observed with using sutures and staples. However, direct tissue damage can still occur. In addition, arrays in which the micro-structures are incorporated are relatively rigid compared to bandages, which reduces their elasticity, flexibility, and conformability, each of which can lead to other clinical problems.

Therefore, wound closure devices containing micro-structures have certain features that are suboptimal for wound closure. First, micro-structures may cause skin irritation when they are inserted in the skin. Both micro-structures and the arrays in which they are incorporated are inherently stiffer, less flexible, and less elastic than the adhesive bandage backing in which they are incorporated. The inelasticity can lead to skin irritation when skin motion occurs, and the skin rubs against the inelastic micro-structure arrays. Second, the inelastic micro-structures themselves can be painful when the skin moves as the micro-structures can move in and out of the skin during movement. Third, micro-structures pierce the skin providing potential entry points for bacteria and other infectious agents. Fourth, the lack of elasticity and flexibility of micro-structure arrays limits their uses over flexor surfaces such as joints. The arrays are prone to detachment when joints bend or in other parts of the body where there is significant skin movement. Skin irritation is a major problem with other less flexible wound closure devices as well. Fifth, the micro-structure arrays reduce the areas in which the adhesive backing contacts the skin which can reduce adherence of the device to the skin. Finally, the micro-structure arrays have inelasticity that differs from the bandage to which it is attached. This can result in inconsistent tension that the device generates along the length of the wound. Focal points of tension lead to inflammation and scarring. An adhesive backing provides more uniform tension along the wound and has been shown to reduce scarring.

To help address these issues, springs can be incorporated into the micro-structure arrays that provide the device with the ability to stretch and flex. This improves the function of the device, but does not entirely solve the problems as elasticity, flexibility, and conformability still remain somewhat limited. In addition, micro-structure density in the wound closure device remains unchanged and thus the clinical issues associated with these structures are not addressed.

The challenge is to create a wound closure device incorporating micro-structures that maintains its strength (e.g., ability to adhere to the tissue and ability to pull a wound closed), while achieving the conformability, flexibility, and elasticity required to close the broad range of wounds encountered in clinical practice. [001I] The present inventors have discovered that micro-structures can be placed along a device at large distances from one another and similar tension is achieved to that which occurs when the devices that include micro-structure arrays are attached immediately adjacent to one another, or very close to each other. The micro-structure arrays configured in such a manner as described herein are able to close wounds by generating sufficient and uniform tension not only over the areas in which they are located but extending throughout the adhesive backing (e.g. a backing, base or substrate to which adhesive is applied) that is located between the two arrays. The design requires fewer micro-structures and their associated arrays thus reducing the clinical problems associated with micro-structure arrays (reduced pain, inflammation, scarring, risk of infection). In addition, more uniform tension along the wound edges reduces inflammation that leads to scarring. By maximizing the area of the wound closure device that contains only adhesive backing relative to the areas containing micro-structure arrays, conformability, elasticity, and flexibility of the device is increased. This is especially advantageous when the base is stretchable or elastic, as a local zone of low stretchability is created over the wound for wound closure and with the rest of the device stretchable to eliminate inflammation or to improve comfort and conformability. It can thus be used in areas that require high levels of device conformability, elasticity, and flexibility for successful wound closure.

Increased conformability allows micro-structure containing wound closure devices to be used in areas that have skin surfaces that are not flat surfaces, such as skin folds including inframammary folds and pannus, the umbilicus, antecubital fossa, posterior surface of the knee, ankles, feet, hands, toes, nose, ears, and fingers. Increased elasticity of micro-structure containing wound closure devices will not only result in better wound closure in general, but especially in areas associated with significant skin movement, such as flexor or extensor surfaces, such as joints, including knees, hips, shoulders, fingers, and toes. Devices with these features, such as those described herein, are also important for successful closing of wounds over areas associated with significant edema.

In examples, large distances between adjacent micro-structure arrays where such benefits can occur can define an elongate wound closure space and can include side-to-side or longitudinal (relative to the length of the wound) distances that are greater than the spacing of micro-structure arrays across a bridge of a micro-structure array, as is described in greater detail below. In examples, large distances between adjacent micro-structure arrays where such benefits can occur can define an elongate wound closure space and can include side-to-side or longitudinal (relative to the length of the wound) distances that are greater than the spacing of micro-structure arrays from edges of the adhesive backing that extend transverse to the wound, as is described in greater detail below.

In an example, a wound closure device comprises a backing comprising a width configured to extend along at least a portion of a wound from a first end to a second end and a length configured to extend transversely across the wound, an adhesive layer attached to the backing, a first micro-structure array attached to the backing proximate the first end, the first micro-structure array configured to extend transversely across the wound, a second micro-structure array attached to the backing proximate the second end, the second micro-structure array configured to extend transversely across the wound, and an elongate wound closure space between the first and second micro-structure arrays.

### BRIEF DESCRIPTION OF TUE DRAWINGS

The patent or application file contains at least one drawing executed in color. Copies of this patent or application publication with color drawing(s) will be provided by the Office upon request and payment of the necessary fee.

FIG. 1 is a plan view of first and second small-sized wound closure devices positioned proximate each other relative to a schematic representation of a wound,

FIG. 2 is a plan view of first and second wide-sized wound closure devices positioned proximate each relative to a schematic representation of a wound.

FIG. 3 is a perspective view of a bottom of the first wide-sized wound closure device of FIG. 2 showing micro-structure arrays extending from a backing.

FIG. 4 is a bottom view of the first wide-sized wound closure device of FIG. 3 showing section plane 5A-5A.

FIG. 5A is a side cross-sectional view of the first wide-sized wound closure device of FIG. 4 showing a micro-structure and a slit.

FIG. 5B is a close-tip cross-sectional view of the first wide-sized wound closure device of FIG. 5A showing the micro-structure of the micro-structure array projecting outward.

FIG. 5C is a close-up cross-sectional view of the first wide-sized wound closure device of FIG. 5A showing the slit extending through the backing.

FIG. 6 is a color finite element analysis illustration of a wide-sized wound closure device of FIG. 2 showing even tension applied longitudinally across the device to simulate closure of a wound with the device holding tissue.

FIG. 7 is a color finite element analysis illustration of a wide sized wound closure device of similar to that of FIG. 6, but with the backing to which the micro-structure arrays are attached being cut in half.

FIG. 8A is a color photograph of a wound closure device of the present disclosure comprising widely spaced micro-structure arrays applied to tissue proximate a knee joint.

FIG. 8B is a color photograph of the tissue of FIG. 8A after application of the tissue closure device of the present application for nine days.

FIG. 9A is a color photograph of a previous wound closure device comprising closely spaced micro-structure arrays applied to tissue proximate a knee joint.

FIG. 9B is a color photograph of the tissue of FIG. 9A after application of the previous wound closure device for nine days.

FIG. 10A is a color photograph of a previous wound closure device comprising closely spaced micro-structure arrays applied to tissue proximate a knee joint.

FIG. 10B is a color photograph of the tissue of FIG. 10A after the application of the previous wound closure device for twelve days.

FIG. 11A is a plan view of a wound closure device of the present disclosure incorporating more than two micro-structure arrays at spaced intervals to produce elongate wound closure spaces.

FIG. 11B is a perspective view of the wound closure device of FIG. 11A.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

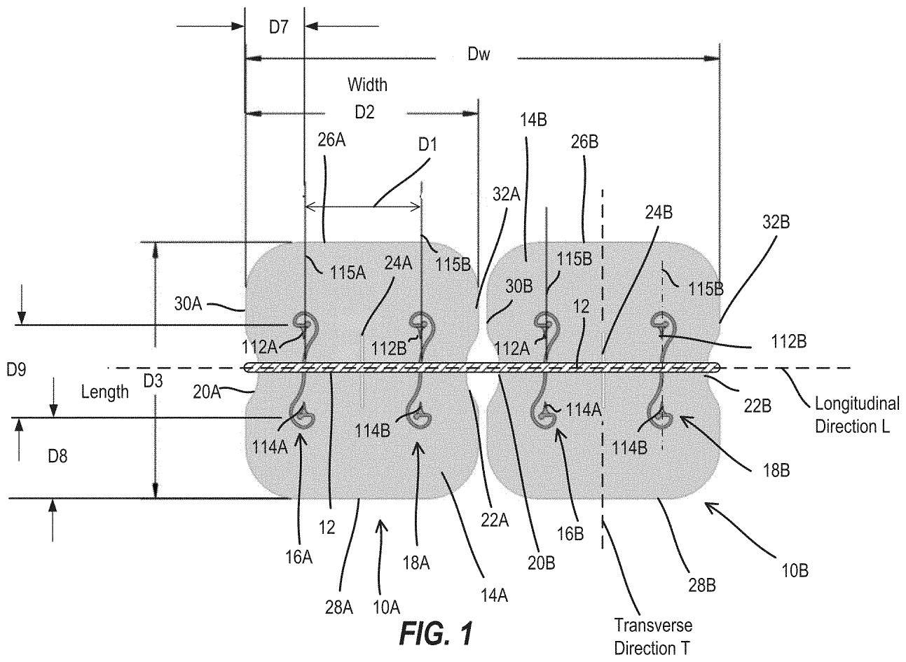

FIG. 1 is a plan view of first wound closure device 10A and second wound closure device 10B positioned proximate each other relative to a schematic representation of wound 12. Wound 12 is schematically illustrated as being located across a portion of both wound closure devices 10A and 10B. Wound closure device 10A can comprise elastic backing 14A, micro-structure array 16A and micro-structure array 18A. Wound closure device 10B can comprise an elastic backing 14B, micro-structure array 16B and micro-structure array 18B. Backing 14A can comprise first indentation 20A, second indentation 22A and slit 24A, each of which can be an optional feature. Backing 14B can comprise first indentation 20B, second indentation 22B and slit 24B, each of which can be an optional feature. Backing 14A can extend lengthwise between sides 26A and 28A and widthwise between sides 30A and 32A. Backing 14B can extend lengthwise between sides 26B and 28B and widthwise between sides 30B and 32B. Wound closure device 10B can be configured identically as wound closure device 10A and thus all dimensions referenced on either one of devices 10A and LOB applies to the other as well.

Axes 15A and 115B of micro-structure array 16A and micro-structure array 18A can be spaced apart distance D1. Backing 14A can comprise axial width D2, which can extend in longitudinal direction L relative to wound 12, and axial length D3, which can extend in transverse direction T relative to wound 12. As such, width D2 can be considered to extend in the longitudinal direction, and length D3 can be considered to extend in the transverse direction. Wound closure device 10B can be configured similarly as wound closure device 10A. Though wound closure devices 10A and 10B are described with a particular orientation for “length” and “width” of the devices, the length of the device may actually be shorter than the width of the device using the dimensions such that “length” does not always refer to the longest dimension.

Wound closure device 10B can be configured to be placed proximate to wound closure device 10A to close wounds longer than width D2 of wound closure device 10A. As such, multiple wound closure devices can be used next to each other to close wounds longer than an individual wound closure device itself. Thus, the total distance Dw of the two adjacent wound closure devices 10A and 10B can be approximately equal to or slightly larger than twice D2.

With reference to FIG. 1, one of wound closure devices 10A and 10B can be used alone to close a wound. In the illustrated example, wound 12 of FIG. 1 can extend across micro-structure array 16A and micro-structure array 18B to edges 30A and 32B. In examples, wound closure devices 10A and 10B can be configured to each close wound up to about 1.0 inch (˜2.54 cm), which is approximately equal to D2. Two of wound closure devices 10A and 10B can be used together to close a wound up to about 2.0 inches (˜5.1 cm). As such, wound 12 can have a length that extends from anywhere between micro-structure arrays 16A and 18A to anywhere between edges 30A and 32B, or other lengths. However, in other examples, a single wound closure device 10A or 10B can be used to close wounds wider than D2 and two of wound closure devices 10A and 10B can be used to close wounds wider than Dw. In examples, D1 can be approximately 0.5 inches (˜1.27 cm). In examples, D3 can be approximately 1.1 inches (˜ 2.8 cm).

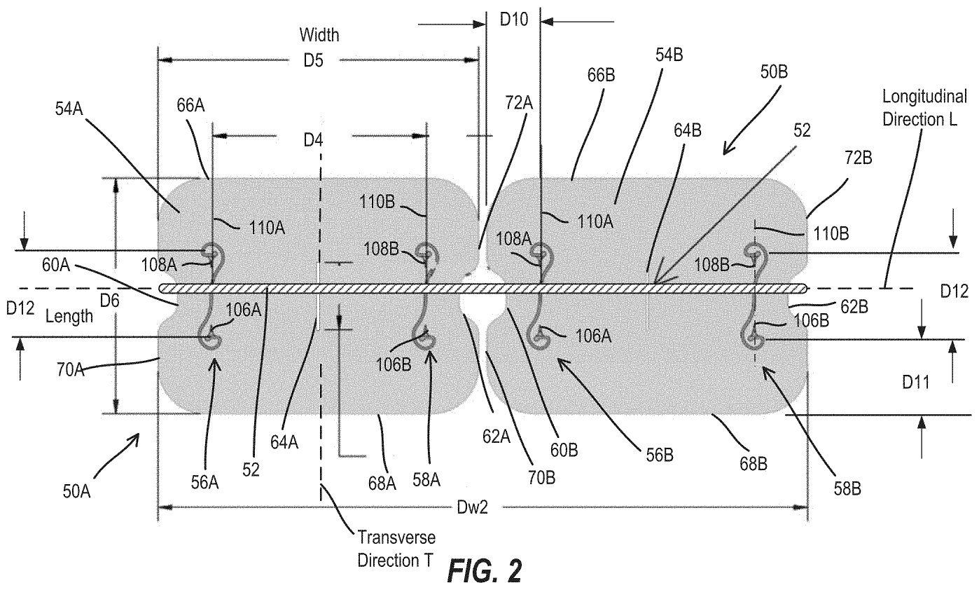

FIG. 2 is a plan view of first wound closure device 50A and second wound closure device 50B positioned proximate to each other relative to a schematic representation of wound 52. Wound closure device 50A can comprise elastic backing 54A, micro-structure array 56A and micro-structure array 58A. Wound closure device 50B can comprise elastic backing 54B, micro-structure array 56B and micro-structure array 58B. Backing 54A can optionally comprise first indentation 60A, second indentation 62A and slit 64A. Backing 54B can optionally comprise first indentation 60B, second indentation 62B and slit 64B. Backing 54A can extend lengthwise between sides 66A and 68A and widthwise between sides 70A and 72A. Backing 54B can extend lengthwise between sides 66B and 68B and widthwise between sides 70B and 72B. Wound closure device 50B can be configured identically as wound closure device 50A and thus all dimensions referenced on either one of devices 50A and 50B applies to the other as well.

Axes 110A and 110B of micro-structure array 56A and micro-structure array 58A can be spaced apart at distance D4. Backing 54A can comprise axial width D5, which can extend in longitudinal direction L relative to wound 52, and axial length D6, which can extend in transverse direction T relative to wound 52. As such, width D5 can be considered to extend in the longitudinal direction, and length D6 can be considered to extend in the transverse direction, Wound closure device OB can be configured similarly as wound closure device 50A. Though wound closure devices 50A and 50B are described with a particular orientation for “length” and “width” of the devices, the length of the device may actually be shorter than the width of the device using the dimensions such that “length” does not always refer to the longest dimension.

Wound closure device 50B can be configured to be placed proximate to wound closure device 50A to close wounds longer than width D4 of wound closure device 50A. As such, multiple wound closure devices can be used next to each other to close wounds longer than an individual wound closure device itself. Thus, the total distance Dw2 of the two adjacent wound closure devices 50A and 50B can be approximately equal to or slightly larger than twice D4.

With reference to FIG. 2, one of wound closure devices 50A and 50B can be used alone to close a wound. In the illustrated example, wound 52 of FIG. 2 can extend across micro-structure array 56A and micro-structure array 58B to edges 70A and 72B. In examples, wound closure devices 50A and 50B can be configured to each close a wound tip to about 1.5 inches (˜3.8 cm), which is approximately equal to D5. Two of wound closure devices 50A and 50B can be used together to close a wound up to about 3.0 inches (˜7.6 cm). Other combinations and numbers of wound closure devices 10A, 10B, 50A and 50B can be used to achieve the desired length of wound closure. As such, wound 52 can have a length that extends from anywhere between micro-structure arrays 56A and 58A to anywhere between edges 70A and 72B, or other lengths. However, in other examples, a single wound closure device 50A or 50B can be used to close wounds wider than D5 and two of wound closure devices 50A and 50B can be sued to close wounds wider than Dw2. In examples, D4 can be approximately 1.0 inch (˜2.54 cm). In examples, D6 can be approximately 1.1 inches (˜2.8 cm).

With reference to FIGS. 1 and 2, each of wound closure devices 10A, 10B, 50A and 50B is configured to be placed over a wound, e.g., a laceration, in tissue, e.g., the epidermis. In examples, wound closure devices 10A, 10B, 50A and 50B can be used to treat skin incisions, port sites, or other wounds on a knee, a hip, an abdomen, a hand, fingers, foot, toes, a head, neck, pelvis, a back, a chest, an extremity, areas of high mobility, flexibility, or anywhere on the body.

Wounds 12 and 52 can be linear, curvilinear, or jagged. In comparison to wound closure devices 50A and 50B, wound closure devices 10A and 10B (FIG. 1) can be used to treat shorter wounds and wound closure devices 50A and 50B (FIG. 2) can be used to treat longer wounds, like wound 52. A combination of wound closure device 10A and 50A can be used to treat wounds of intermediate lengths. The size of wounds that can be treated by wound closure devices 10A, 10B, 50A and 50B, such as wounds 12 and 52, can extend in different lengths that are shorter than a single backing up to and including all the way across both of two or more of various combinations of backings 14A and 14B and backings 54A and 54B, as discussed herein. Thus, various instances and combinations of wound closure devise 10A, 10B, 50A and 50B can be used in different permutations to close wounds of different lengths. As shown in FIGS. 1 and 2, wound closure devices 10A, 10B, 50A and 50B can be used in multiples to cover wounds longer than an individual device is wide. Width D2 (FIG. 1) and width D5 (FIG. 2) can extend in the direction substantially parallel to the wound, in the longitudinal direction. The length D3 of wound closure device 10A in FIG. 1 and the length D6 of wound closure device 50A in FIG. 2 extend transverse to the widths of backings 14A and 54A, respectively. Backing 14A and backing 54A can be configured to stretch such that D2 and D3 (FIG. 1) and D5 and D6 (FIG. 2) can vary after application and wear on the skin, but is typically constant (at rest, e.g., when not subject to tension from a wound or compression from a micro-structure array). Backing 14A and backing 54A can generally form a rectilinear, e.g., rectangular shape, except for some of the edge features described herein, such as rounding of the corners and indentations 20A and 22A and indentations 60A and 62A. Dimensions D3 and D6 extend generally orthogonally to the widths D2 and D5 of wound closure devices 10A and 50A and transverse to or orthogonal to wounds 12 and 52, respectively. Backings 14A, 14B, 54A and 54B can have other shapes, including, but not limited to, square, circular, oval, oblong and the like. The present inventors have found that backings having elongate aspect ratios, such as rectangular, oval or otherwise oblong are particularly well-suited to form elongate wound closure spaces described herein.

Furthermore, wound closure devices 10A, 10B, 50A and 50B in FIGS. 1 and 2 can be used in conjunction with other wound closure devices such as staples, sutures, tissue adhesive or glue, bandages and the like. For example, wound closure devices 10A, 10B, 50A and 50B can be used with the wound closure devices described in US Patent Publication Nos. 2015/0305739 and 2017/0333039, previously incorporated herein by reference and attached as Appendix A and Appendix B. In an example application, devices described in US Patent Publication No. 2017/0333039 can be used alongside one or more devices of the present disclosure. For example, concurrent use of staples and sutures with the devices of the present disclosure can provide a wound barrier and uniform tension reducing infection and scarring, respectively.

The wound closure devices can be applied immediately adjacent to one another to completely seal the wound or be separated purposely with a small gap to allow drainage, breathing and the like. For example, with reference to FIG. 2, edge 72A can be placed in contact with edge 70B. As discussed below, indentations 62A and 60B can permit egress of fluid from wound 52. However, the edges of the device can also be designed so as not to have indentations 62A and 60B such that edges 72A and 70B are linear without a curve in the in the middle of the ends. This can enable a tight wound seal, which can further reduce the risk of bacterial entry into the wound. In examples, backings 54A and 54B can include indentations 60A and 60B, respectively, and omit indentations 62A and 62B so that an indentation opening can be formed or omitted depending on the orientations of backings 54A And 54B as applied relative to each other.

Wound closure devices 10A, 10B, 50A and 50B in FIGS. 1 and 2 can be configured per the present disclosure to provide even tension across the longitudinal length of the wound when it is closed (thereby promoting healing, reducing the possibility of infection, and reducing inflammation and scarring) while simultaneously minimizing direct irritation to the skin (e.g., fewer insertion points from micro-structures into the skin and reduced number of arrays interacting with the skin), decreasing the number of ports of entry of bacteria through insertion sites due to decreased number of micro-structures, and increasing conformability, elasticity, and flexibility (such as by reducing the number of attachment points into the skin and reducing the amount of stiff material, such as metal, in the device). This will reduce irritation and scarring resulting from inflammation and tissue damage. It will also decrease the risk of the wound closure device detaching from the skin that occurs with skin motion such as over flexor surfaces like joints. For example, stiff material within a wound closure device can cause the wound closure device to buckle when the skin moves, thereby potentially causing the device to dislodge from engagement with skin. This design allows use of less stiff materials to minimize irritation while creating low stretchability/high tension areas for wound closure.

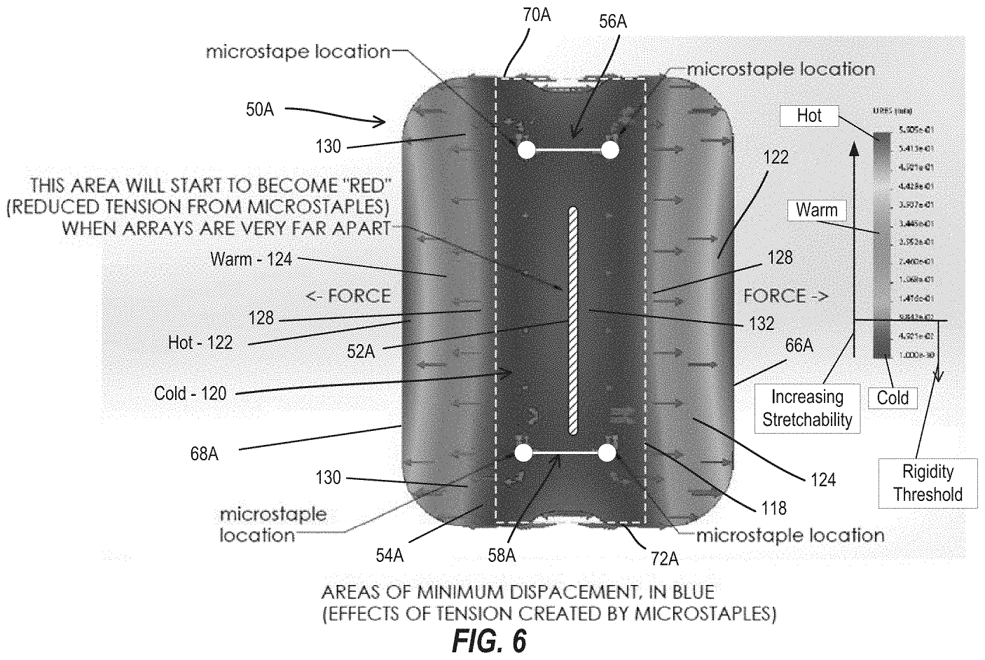

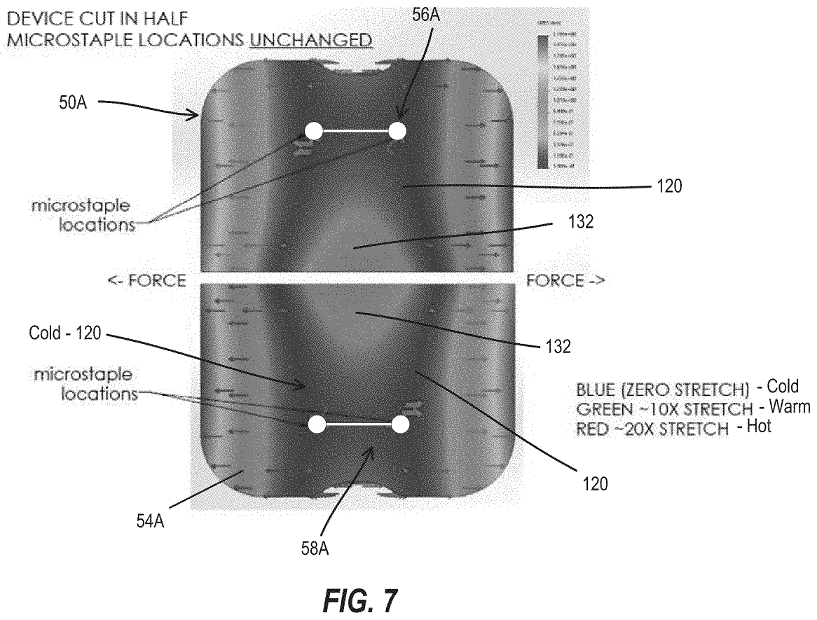

With continued reference to FIGS. 1 and 2, wound closure devices 10A, 10B, 50A and 50B can be configured such that micro-structure arrays on each device are positioned relative to the dimensions of backings 14A, 14B, 54A and 54B, respectively, such that two micro-structure arrays (e.g., micro-structure arrays 16A and 18A, micro-structure arrays 16B and 18B, micro-structure arrays 56A and 58A, and micro-structure arrays 56B and 58B) can be positioned proximate to two longitudinally spaced edges (e.g., edges 60A and 62A, edges 60B and 62B, edges 70A and 72A, and edges 70B and 72B) of the wound closure device to apply tension adequately and approximately evenly across the entire width of the device (D2 of FIG. 1 or D5 of FIG. 2), as is discussed in greater detail below, particularly with reference to FIGS. 6 and 7.

As discussed herein, wound closure devises 10A, 10B, 50A and 50B can be configured to close wounds extending along the widths of backings 14A, 14B, 54A and 54B, respectively, without having to place micro-structure arrays across the wounds at close longitudinally spaced intervals to one another. Thus, the space between micro-structure array 16A and 18A on backing 14A (FIG. 1), the space between micro-structure array 16B and 18B on backing 14B (FIG. 1), the space between micro-structure arrays 56A and 58A on backing 54A (FIG. 2), and the space between micro-structure arrays 56B and 58B on backing 54B (FIG. 2) can define a portion of backings 14A, 14B, 54A and 54B, respectively, comprising elongate wound closure spaces defined herein as portions of backings 14A, 14B, 54A and 54B between immediately adjacent (e.g., next to but not necessarily touching) microstructure arrays configured to pull or hold a wound closed, not only in the transverse direction at the longitudinal location in which the micro-structure arrays are located, but in the longitudinal space between the micro-structure arrays. As discussed below with reference to FIGS. 6 and 7, the resiliency or elasticity of backings 14A, 14B, 54A and 54B can allow the transverse holding strength of micro-structure arrays 16A-58B to be expanded longitudinally into the elongate wound closure spaces. In examples of FIG. 1, elongate wound closure space of wound closure devices 10A and 10B can be defined as the spaced occupied within D2 and D9. In examples of FIG. 2, elongate wound closure space of wound closure devices 50A and 50B can be defined as the space occupied within D5 and D12, which is referenced as cold (blue) zone 120 in FIG. 6.

As can be seen in FIG. 1, micro-structure array 16A can be positioned on backing 14A proximate to edge 30A and micro-structure array 18A can be positioned on backing 14A proximate edge 32A. Micro-structure array 16A (as defined by a central axis 115A) can be spaced distance D7 from edge 30A. Micro-structure array 18A can be spaced a similar distance from edge 32A as distance D7. Distance D7 which can comprise a first distance and distance D2 (FIG. 1) can comprise a second distance where the second distance is greater than the first distance. As such, distance D7 can, at least partially, define an elongate wound closure space.

Micro-structure array 16A can be positioned so that micro-structure 114A is positioned distance D8 from edge 28A. Micro-structure 112A can be positioned a similar distance from edge 26A as distance D8. Likewise, micro-structures 112B and 114B can be spaced from edges 26A and 28A, respectively, similar to distance D8. Positioned as such, micro-structures 112A-114B can be close to the rounded corners of backing 14A to provide gripping and tensioning functionality to the entirety of backing 14A, while still providing the elongate wound closure space.

In examples, distance D7 can be approximately 0.25 inches (˜6.4 mm). In examples, distance D8 can be approximately 0.3 inches (˜7.6 mm). Micro-structure array 16A can have distance D9 between micro-structures 112A and 114A. In examples, distance D9 can be approximately 0.5 inches (˜12.7 mm). Micro-structure arrays 16A and 18A and micro-structure arrays 16B and 18B can be placed in like manners on backings 14A and 14B, respectively.

As can be seen in FIG. 2, micro-structure array 56B can be positioned on backing 54B proximate to edge 70B and micro-structure array 58B can be positioned on backing 54B proximate edge 72B. Micro-structure array 56B (as defined by a central axis 110B) can be spaced distance D10 from edge 70B. Micro-structure array 58B can be spaced a similar distance from edge 72B as distance D10. Distance DIG which can comprise a first distance and distance D5 (FIG. 2) can comprise a second distance where the second distance is greater than the first distance. As such, distance DIG can, at least partially, define an elongate wound closure space.

Micro-structure array 58B can be positioned so that micro-structure 106B is positioned distance D11 from edge 68B. Micro-structure 108B can be positioned a similar distance from edge 66B as distance D11. Likewise, micro-structures 108A and 106A can be spaced from edges 66B and 68B similar to distance D1, Positioned as such, micro-structures 106A-108B can be close to the rounded corners of backing 54B to provide gripping and tensioning functionality to the entirety of backing 54B, while still providing the elongate wound closure space.

In examples, distance DIG can be approximately 0.25 inches (˜6.4 mm). In examples, distance D11 can be approximately 0.3 inches (˜7.62 mm). Micro-structure array 58B can have distance D12 between micro-structures 108B and 106B. In examples, distance D12 can be approximately 0.5 inches (˜12.7 mm). Micro-structure arrays 56A and 58A and micro-structure arrays 56B and 58B can be placed in like manners on backings 54A and 54B, respectively.

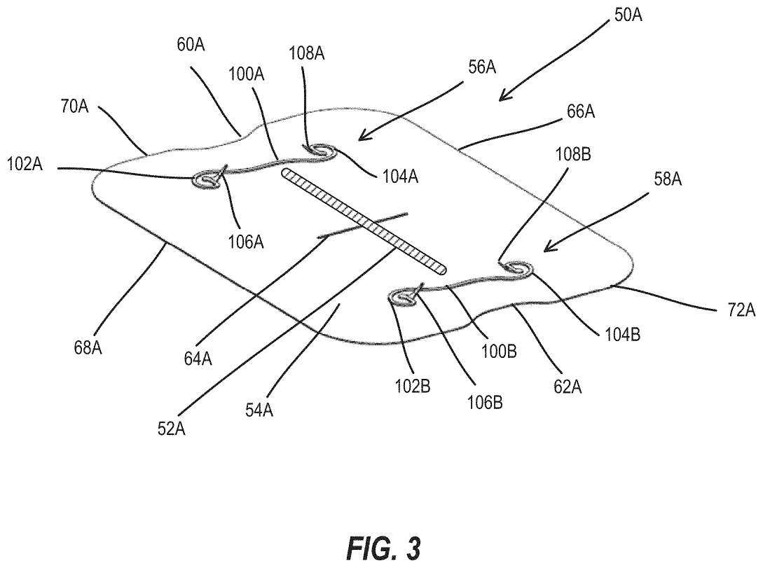

FIG. 3 is a perspective view of a bottom or wound-facing side of wound closure device 50A having micro-structure arrays 56A and 58A disposed on backing 54A. Micro-structure array 56A can comprise bridge 100A, spring structure 102A, spring structure 104A, micro-structure 106A and micro-structure 108A. Micro-structure array 58A can comprise bridge 100B, spring structure 102B, spring structure 104B, micro-structure 106B and micro-structure 108B. A plurality of micro-structures can define an array. Micro-structures 106A and 108A can comprise any micro-sized structure suitable for grabbing onto or piercing into tissue, e.g., skin, such as barbs, hooks, anchors, needles, blades, fishscales, pillars, hairs (i.e., a microstaple, a microbarb, a microneedle, a microblade, a microanchor, a microhook, a microfishscale, a micropillar, and a microhair) and the like.

Micro-structure arrays 16A, 16B, 18A, 18B, 56B and 58B can be configured similarly as micro-structure arrays 56A and 58A, though numbering and discussion is omitted for brevity.

Backings 14A, 14B, 54A and 54B can be configured similarly as each other, with backings 14A and 14B having different widths than backings 54A and 54B. Exemplary features of backings 14A, 14B, 54A and 54B will be described with reference to backing 54A and FIG. 3 and wound 52A.

Backing 54A can comprise a stretchable/elastic substrate or base upon which other components of wound closure device 50A can be affixed. Backing 54A can be any material such as a fabric or polymer. In some examples, backing 54A can comprise a material singularly, or in combination, selected from the group consisting of medical tape, white cloth tape, surgical tape, tan cloth medical tape, silk surgical tape, clear tape, hypoallergenic tape, silicone, elastic silicone, polyurethane, elastic polyurethane, polyethylene, elastic polyethylene, rubber, latex, expanded PTFE (ePTFE), plastic and plastic components, polymers, biopolymers, and natural materials. In examples, backing 54A can comprise a silicone sheet. In examples, devices of the present disclosure can include bases, backings or substrates made in similar to those structures disclosed in US Patent Publication Nos. 2015/0305739 and 2017/0333039, previously incorporated herein by reference and attached as Appendix A and Appendix B. However, as disclosed herein, it can be advantageous to have bases, backings or substrates shaped as described herein to, for example, permit micro-structure arrays located proximate to longitudinal edges 70A and 72A of rectilinear backings transmit wound closure forces of the micro-structure arrays to elongate wound closure space between first and second micro-structure arrays.

With reference to FIG. 3, backing 54A can comprise a sheet configured to stretch when subject to a tensile load but that will return to its original shape (or close to it) when loading is removed. During use applied to skin, backing 54A may be unstretched, partially stretched or completely stretched, Backing 54A can comprise a continuous material that covers wound 52 to help seal the wound and prevent infection. In this example, the outer-most corners of backing 54A can be rounded with a large radius in order to avoid having sharp ninety-degree corners. This helps reduce the chance that the sharp corners will catch on clothing or other objects causing detachment of the device away from the skin. Sides 66A and 68A of device 50A can extend parallel to a longitudinal direction (i.e., parallel to wound 52A) to define the width of device 50A and can generally be linear, although they may be any shape. In examples, having sides 66A and 68A be straight and parallel can help uniformly distribute wound closure forces to wound 52A. Sides 70A and 72A of device 50A can be parallel to the length of device 50A (i.e., transverse to wound 52A) and can generally be linear, although they may be any shape.

An adhesive layer or adhesive backing can be disposed on the tissue-facing side to allow wound closure device 50A to be secured to tissue in conjunction with micro-structure arrays 56A and 58A. The adhesive can be applied uniformly across the entire backing or only around the perimeter of the backing (e.g., backing 54A) to ensure good adhesion and sealing to the wound area or interspersed throughout the backing. Other adhesive patterns can also be used and in this application. The teams adhesive, adhesive layer and adhesive backing are used interchangeably. Micro-structure arrays 56A and 58A can be applied to the tissue-facing side against the adhesive layer. The adhesive can include any medical grade adhesive, such as, for example, an acrylate hydrocolloid or silicone.

Backing 54A can include indentations 60A and 62A and slit 64A, which can be used to provide additional features and functionality to wound closure device 50B.

As can be seen in FIG. 3, indentations 60A and 62A can comprise disruptions in the linear nature of edges 70A and 72A that can create space between adjacent wound closure devices, such as devices 50A and 50B. Indentations 60A and 62A can be configured to allow passage of matter into and out of wound 52A if length of the wound extends up to or beyond 60A and 62A, such as air and fluid, respectively. In the illustrated example, indentations 60A and 62A have undulating edges, resulting in scallops. In other examples, indentations 60A and 62A can comprise square or rectangular indentations along the edge would result in a castellated edge.

In examples, backing 54A can include one or more slits 64A (sometimes also referred to as an aperture or a slot) extending through backing 54A. Slit 64A can be configured to allow drainage of fluid from wound 52A. In the illustrated example, there is a single slit 64A proximate the center of backing 54A and two indentations 60A and 62A on either side of backing 54A, although there may be more or less. Alternatively, backing 54A may contain no slits to allow sealing of the wound if wound length is less than the distance between 60A and 62A. This is important in certain clinical settings where entry of bacteria or other infectious agents is a major clinical issue. A single slit 64A is provided in a direction parallel to the lengths of micro-structure arrays 56A and 58A, parallel to the length of device 50A (e.g., along axes 110A and 110B of FIG. 2). Slit 64A can be positioned proximate (e.g., on top of) wound 52A to facilitate drainage. Slit 64A can have a variety of shapes, including rectangular (as illustrated), square, oval, elliptical, circular, round, etc. and combinations thereof.

Additionally, backing 54A can be formed from a stretchable material so that it can be stretched across wound 52A and conform to the wound. In an example, backing 54A can be elastic, e.g., a substance or object able to resume its normal shape spontaneously after contraction, dilatation, or distortion.) Stretching device 50A across the wound can apply a closure force across wound 52A which can facilitate wound closure and healing since the closure force will help appose opposite ends of tissue in wound 52A.

Indentations 60A and 62A and slit 64A can additionally improve flexibility of backing 54A and help backing 54A conform to the contours of the wound as well as allowing an aperture through which fluid from the wound may drain. For example, when a large adhesive bandage is applied to a wound having contours, the adhesive bandage may not always conform to the contours of the anatomy and the bandage may ripple, buckle or tent outward thereby creating additional stress on the adhesive and allowing the device to more easily fall off the patient. Therefore, adding slits to the interior of backing 54A and indentations 60A and 62A to the perimeter of backing 54A, and similar features, can allow the material of the backing to better conform to the native anatomy reducing local stress on adhesive and thus reducing risk of local adhesive failure.

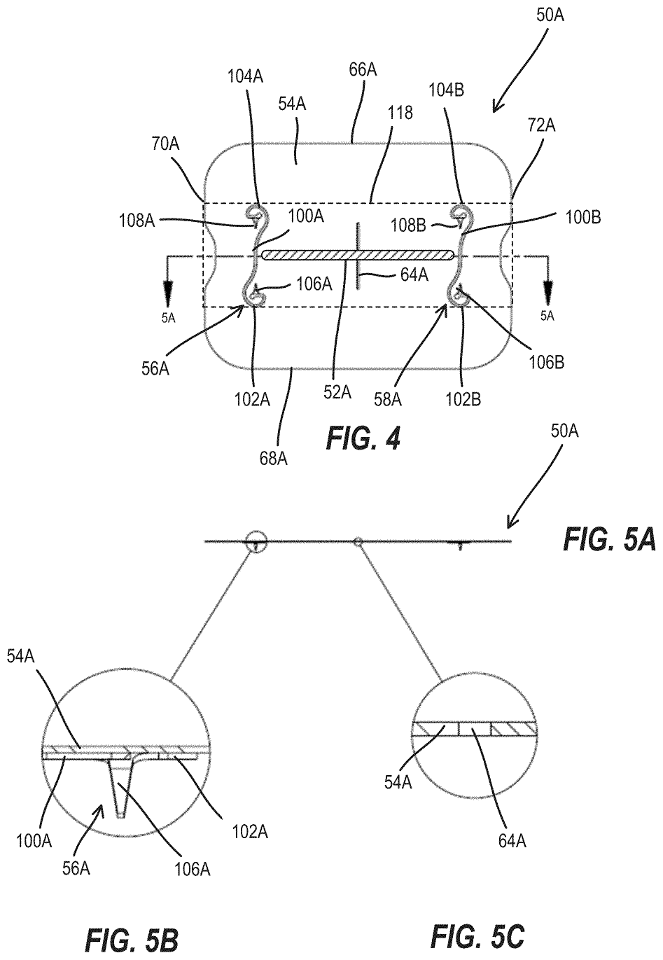

FIG. 4 is a bottom view of first wide-sized wound closure device 50A of FIG. 3 showing section plane 5A-5A. FIG. 5A is a side cross-sectional view of first wide-sized wound closure 50A device of FIG. 4 showing micro-structure 106A and slit 64A. FIG. 5B is a close-up cross-sectional view of first wide-sized wound closure device 50A of FIG. 5A showing micro-structure 106A of micro-structure array 56A projecting outward. FIG. 5C is a close-up cross-sectional view of first wide-sized wound closure device 50A of FIG. 5A showing slit 64A extending through backing 54A. FIGS. 4-5C are discussed concurrently.

In FIG. 4, micro-structure arrays 56A and 58A are shown configured to have only a single bridge (110A or 100B) and a single micro-structure (106A, 106B, 108A, 108B) disposed at each end of the bridge via a single spring structure (102A, 102B, 104A, 104B). In additional examples, micro-structure arrays having one or both of multiple bridges and multiple micro-structures at each end of the bridge can be used. In examples, devices of the present disclosure can include micro-structure arrays generally having a form similar to micro-structure arrays disclosed in US Patent Publication Nos. 2015/0305739 and 2017/0333039, previously incorporated herein by reference and attached as Appendix A and Appendix B. However, as disclosed herein, it can be advantageous to minimize the number of bridges and micro-structures used to, for example, maximize elasticity of the wound closure device, which will minimize inflammation caused by the wound closure device, respectively and increase the conformability of the wound closure device, as is discussed herein.

As shown in FIG. 4, wound closure device 50A can include two micro-structure arrays 56A and 58A. In various examples, wound closure device 50A is configured to have only two micro-structure arrays such that elongate wound closure space 118 is provided between micro-structure arrays 56A and 58A, thereby accruing the benefits described herein. Thus, for example, space along backing 54A between first and second micro-structure arrays 56A and 58A can be uninterrupted with another micro-structure array such that longitudinal tension of the first and second micro-structure arrays can be uninterrupted, as described below with reference to FIGS. 6 and 7. In examples, elongate wound closure space 118 between micro-structure arrays 56A and 58A can be greater than the distance between micro-structure array 56A and edge 70A of backing 54A, and can be greater than the distance between micro-structure array 58A and edge 72A of backing 54A. In examples, the elongate wound closure space between micro-structure arrays 56A and 58A can be greater than a distance between micro-structure 108A and micro-structure 106A in each of micro-structure arrays 56A and 58A.

As shown in FIG. 4, bridge 100A can comprise a strut configured to extend in a generally straight manner between spring structures 102A and 104A and can be sized to extend across the height (e.g., transverse thickness) of wound 52A. In other examples, bridge 100A can be sinusoidal or have an otherwise undulating shape that can stretch outward under tension. Bridge 100A can also be curved or jagged. Spring structures 102A and 104A can comprise curved or undulating lengths of material shaped to allow ends of array 56A to extend further away from bridge 10A, thereby also moving micro-structures 106A and 108A further away from one another when tension is applied thereacross. Micro-structures 106A and 108A can move back to their original position or rest position when the tension is removed. In the illustrated example, spring structures 102A and 104A can be spiral shaped, nautilus shaped or semi-circular shaped. Jagged or curved shapes can also be used. Thus, the material defining spring structures 102A and 104A can have shapes that provide resiliency and stretchability to micro-structure array 56A.

FIG. 4 shows micro-structure arrays 56A and 58A comprising only a single micro-structure 106A, 108A, 106B and 108B at each end of devices 56A and 56B. In other examples, any number of micro-structures can be used. However, the present inventors have found that adequate effects of micro-structures (e.g., ensuring adequate attachment to tissue and providing closure to a wound) can be achieved with only a total of four micro-structures in the device (two micro-structures in each of two arrays). In examples, the four micro-structures, such as micro-structures 106A, 108A, 106B and 108B can be distributed proximate the four corners of the rectilinear shape of backing 54A.

Micro-structure array 56A can be formed of any material including metals, polymers, or other materials known in the art. In examples, micro-structure array 56A can be made of aluminum, titanium, and stainless steel including 300 series stainless steel alloys and 316 stainless steel alloys. In examples, micro-structure array 56A can be made of poly(methyl methacrylate) (also known as Poly(methyl2-methylpropenoate (IUPAC name), polymethyl methacrylate, or more commonly known as PLEXIGLASS™), silicon, and chitin. Micro-structure array 56A can comprise a single, monolithic piece of material. Micro-structure array 56A can be formed from a sheet of material that is stamped or etched to the desired shape and then bent to form micro-structures or spring structures.

As shown in FIG. 5B, micro-structures 106A and 108A can comprise protrusions extending from spring structures 102A and 104A and can have a tissue piercing tip that can extend into tissue to anchor wound closure device 50A to skin of a patient. The depth of penetration of micro-structures 106A and 108A can be deep enough to ensure adequate retention strength yet not to a depth that pain, inflammation, scarring, etc. become an issue. Micro-structures 106A and 106B can be perpendicular to adhesive backing 54A or preferably, less than 90 degree angle with respect to backing 54A, and most preferably at a 45 degree angle to backing 54A.

Wound closure device 50A can include other components such as, but not limited to, nanostructures (e.g., nanostructure arrays or nanofibers) and bioactive compounds (e.g., drugs, therapeutics, hydrogels, healing substances, and combinations thereof). In some implications, the material is selected from the group consisting of PMMA, silicone, chitin, chitosan, ecoflex, titanium, glass, metal, steel, silicon, silk, catgut, chromic catgut, polyglycolic acid, polydioxanone, polytrinethulene carbonate, nylon, polypropylene, polyester, polybutester, poly(lactic-co-glycolic acid), polylactone, elastin, resilin, collagen, cellulose, polymers of hydroxy acids such as lactic acid and glycolic acid polylactide, polyglycolide, polylactide-co-glycolide, and copolymers with PEG, polyanhydrides, poly(ortho)esters, polyurethanes, poly(butyric acid), poly(valeric acid), and poly(lactide-co-caprolactone). Representative non-biodegradable polymers include polycarbonate, polymethacrylic acid, ethylenevinyl acetate, polytetrafluoroethylene (TEFLONT), polyesters, and any combination thereof. In some implementations, wound closure device 50B also includes chitin (e.g., chitin nanofibers). In some implementations, the wound closure devices include a hydrogel.

Other aspects of wound closure device 50A are described in and may be included in any of the examples disclosed herein, such as in US Patent Publication Nos. 2015/0305739 and 2017/0333039, previously incorporated herein by reference and attached as Appendix A and Appendix B.

Wound closure devices 10A, 10B, 50A and 50B of the present disclosure can be used with applicator tabs (not illustrated), also referred to as application tabs. The applicator tabs can be releasably coupled to an edge of the device along the width of the device (e.g., extending along edge 66A or 68A of backing 54A), such as to facilitate pulling of the device and stretching in the direction of the length of the device. The applicator tabs may extend along the entire width of the devices, slightly less than the width of the devices, or slightly beyond the width of the devices. The tabs can help provide support to the device to help the operator keep the device generally flat and planar for ease in and more consistent application to the wound. Otherwise, a long flat sheet of material may deform under its own weight and wrinkle or deform and then the adhesive portions may adhere to one another making application to the wound area problematic. The applicator tabs can have elongate projections that extend from the applicator tab and are releasably coupled with the adhesive layer on the backing layer. Thus, the applicator tab helps support the device and also simultaneously optimizes skin surface area contact with the adhesive layer. This allows fine-tuning of the amount of force required for the operator to pull the applicator tab away, and thus creating the intended amount of tension during application. Fine tuning the amount of contact area between the applicator tab and the adhesive layer is also advantageous by minimizing inadvertent removal or peeling away of adhesive from the backing which could affect the adhesion of the device to the wound during its application. The applicator tab may also be tapered to provide a tab that is easy to grasp between a thumb and finger or between fingers and also avoids having sharp corners which could catch on the operator's fingers, surgical gloves, or other adjacent objects. A release liner is coated over the applicator tab such that a portion of the adhesive layer of the backing (14A, 14B, 54A and 54B) is in direct contact while the other portions of the device are on a different release liner from the rest of the packaging (not illustrated). Multiple applicator tabs can be coupled to the device. Or, in some examples a single applicator tab having a sufficient span to extend across the width of the wound closure device along edge 66A or 68A can be used. Optional instructions, size information, warnings or other information may be printed on the applicator tab if desired.

The combined unit can then be disposed in a sterile package as Tyvek pouch, procedure kit or other packaging (not illustrated) and then terminally sterilized. The applicator tab in this example can include instructions or other information such as device size. The release liner can include a series of die-cut windows or apertures extending through the release liner and that are sized and positioned to receive the micro-structure so they are not damaged. The remainder of the release liner can be releasably coupled to the adhesive layer on the backing and can be formed from silicone or another material that is easily peeled away from the adhesive during use, except for the area where the applicator tab is disposed between the adhesive layer and the release liner as previously discussed above.

The configurations of wound closure devices described herein can be used to produce wound closure devices with optimal tensile strength to close a wound. This can be achieved with a device that provides sufficient tensile strength of a wound closure device to provide the external force required to close a wound, while requiring a minimal amount of rigid or non-conformable structures to achieve this rigidity given the clinical problems described above associated with rigid structures. This is achieved with a wound closure device that provides resistance to being displaced by an external force to keep a wound closed all the way across the longitudinal extent of the wound without requiring the use of a large number of micro-structures and micro-structure arrays to provide such tensile strength. Such tensile strength can be useful to overcome forces generated by tissue extending along a wound that have a tendency to open up the wound and stretch the backing. Thus, a backing that is relatively non-stretchable or inelastic can be useful to maintain even tension across a wound, whereas low rigidity (or high stretchability) of a backing can be detrimental to maintaining even tension across a wound. It is noted that rigidity of the backing of the device required to apply the desired amount of closure to the wound is less than the rigidity of a micro-structure array. Consequently, the overall device is able to close a wound without requiring rigid micro-structures to be placed close to one another. The result is a wound closure device with increased conformability, elasticity, and flexibility. FIGS. 6 and 7 show examples of displacement diagrams created by engineering simulation software illustrating desirable tensile strength or rigidity of the backing as achieved by micro-structure array placement to maintain even tension across an elongate wound closure space (FIG. 6) versus a backing cut in half (FIG. 7) resulting in low tensile strength or rigidity due to interruption of the ability of the micro-structure arrays to influence the backing.

FIG. 6 is a color finite element analysis (FEA) illustration of a wide-sized wound closure device 50A of FIGS. 2-4 color coded with a heat map resulting from the application of tension uniformly and perpendicularly along wound 52A (FIG. 4) to simulate forces experienced by the device as it maintains closure of the wound. As illustrated by the key along the right side of FIG. 6, a scale of increasing stretchability is shown ranging from blue (low stretch (also referenced as “hot”)) to red (high stretch (also referenced as “cold”)), Alternative terms, such as “dark”, “medium”, and “light” are included in this description to align with grey scale productions of FIGS. 6 and 7. The blue (dark) narrow band (“cold zone”) extending longitudinally across wound closure device 50A between edges 70A and 72A, indicated generally as 120, represents an area of minimum stretch or displacement (or capacity for displacement) of backing 54A. The red (medium) shaded portions (“hot zones”) alongside edges 68A and 66A, indicated generally as 1 represent areas of maximum stretch or displacement (or capacity for displacement) of backing 54A. The green (light) shaded bands (“warm zones”) between cold zone 120 and hot zones 122, indicated generally as 124, represent areas of intermediate displacement (or capacity for displacement) of backing 54A. In an optimized device, elongate wound closure space (e.g., space 118) would be a solid “cold zone” (blue) that is solid or evenly dark (blue) throughout the entire rectangle. Optimization means even tension is experienced uniformly across the longitudinal width of backing 54A (e.g., from edge 70A to edge 72A) and is thereby transferred to the wound, while reducing the number of micro-structure arrays to increase conformance and reducing the number of micro-structures and arrays to decrease irritation. In FIG. 6, micro-structure array 56A and 58A (schematically represented) are spaced far enough apart to reduce micro-structure array numbers and close enough to each other to maintain cold zone 120 close to an ideal elongate wound closure space 118.

The simulation of FIG. 6 indicates that the non-stretchable portions of micro-structure arrays 56A and 58A create local areas of relative tensile strength or rigidity (“cold zone”—blue areas) that overlap to extend longitudinally all the way across the width of backing 54A between edges 70A and 72A. Cold zone 120 extends almost uniformly across the width of backing 54A in the longitudinal direction between edges 70A and 72A of backing 54A, but is entirely within a threshold amount of rigidity (see scale in FIG. 6), as indicated by cold zone 120 (blue areas) being substantially darker than the rest of backing 54A.

As can be seen at the locations of micro-structure arrays 56A and 58A, the cold zone 120 is thickest at micro-structure arrays 56A and 58A, indicating areas of non-stretch, or minimum displacement, which provides a benefit of being able to hold a wound closed. Thus, the peak transverse closing strengths of micro-structure arrays 56A and 58A, indicated generally at 130, are located in close proximity to each of micro-structure arrays 56A and 58A. Micro-structure arrays 56A and 58A are spaced apart to provide elongate wound closure space 118 (thereby reducing the count of micro-structures), resulting in peak transverse closing strengths 130 of micro-structure arrays 56A and 58A not overlapping (as evidenced by narrowing of cold zone 120 at 128 and the presence of warmer zone 132 (lighter blue area), discussed below), but close enough to still result in the cold zone 120 extending uninterruptedly across backing 54A to provide near uniform tension to the wound. Closer to the center of backing 54A cold zone 120 becomes narrower as the effects of peak transverse closure strengths 130 of micro-structure arrays 56A and 58A on backing 54A diminish and are less able to resist the outward force of the tissue, as shown by signs of a lighter zone at the center of cold zone 120 implying small signs of stretch or displacement, indicated at 132 (lighter blue area around wound 52A). In FIG. 6, this lighter zone is not sufficient to noticeably impact the uniformity of wound closure tension. The size of hot zones 122 (red areas) combined with warm zones 124 (green areas) on either side of cold zone 120 grow in size, indicating increasing outward stretch or displacement present from the tissue, and a corresponding reduction in capacity to hold a wound closed, but not enough to diminish wound closing capabilities to maintain near uniform tension. Note, if micro-structure arrays 56A and 58A were brought closer together on backing 54A, zone 132 and narrowing 128 wound reduced in size.

Each of micro-structure arrays 56A and 58A can have a longitudinal influence on backing 54A that extends the transverse wound-closing capability of each of micro-structure arrays 56A and 58A longitudinally into backing 54A to facilitate pulling sides 66A and 68A together. The longitudinal influence can extend longitudinally from peak transverse closing strengths 130 at micro-structure arrays 56A and 58A into elongate wound closure space 118. As such, wound closure device 50A can be configured to have two longitudinally spaced micro-structure arrays with longitudinal wound closing influences on backing 54A that overlap enough to provide lateral wound-closing capability across the width of device 50A between edges 70A and 72A, as indicated by the uninterrupted dark (blue) outer limits of cold zone 120 in FIG. 6. Thus, peak transverse closing strength 130 of micro-structure array 56A does not overlap with peak transverse closing strength 130 of micro-structure array 58A, as is evidenced by the two warm zones 124. As such, placement of a plurality (e.g., more than two) of immediately adjacent micro-structure arrays across the longitudinal width of device 50A can be avoided where the longitudinal influences significantly overlap to, for example, reduce the number of micro-structure insertion points in the skin. In sum, the general uniformity of cold zone 120 (blue area) from edge 70A to edge 72A allows backing 54A to apply generally even tension across a wound to, for example, reduce or avoid inflammation that leads to scarring without the need for more than two micro-structure arrays on backing 54A that would lead to more irritation, inflammation, and scarring.

FIG. 7 is a color FEA illustration of wide sized wound closure device 50A, similar to that of FIG. 6, but with backing 54A to which micro-structure arrays 56A and 58A are attached being cut in half FIG. 7 indicates that the continuity of backing 54A between micro-structure arrays 56A and 58A has been disrupted such that cold zone 120 is disrupted. Thus, FIG. 7 demonstrates that elongate wound closure space 118 of the present application shown in FIG. 6 can be achieved by placing a portion of backing 54A between micro-structure arrays 56A and 58A without the presence of an additional micro-structure array to maintain continuity of cold zone 120 (blue area). In other words, cold zone 120 (blue area) can be maintained between micro-structure arrays 56A and 58A across elongate wound closure space 118 (FIG. 6) that is much larger than the distance between micro-structure arrays 56A and 58A and edges 70A and 72A (e.g., D7 of FIG. 4), respectively. FIG. 7 indicates that the transverse wound closure capability is interrupted by the cut in backing 54A. As such, the ability of backing 54A to provide transverse wound closure capabilities at the cut are diminished, as shown by warm zones 132 (green areas). Furthermore, the ability of micro-structure arrays 56A and 58A to apply uniform tension across each half of backing 54A is impaired due to the presence of only a single micro-structure array, as is evidenced by warm zones 132 (green areas). Warm zones 132 would have the tendency to produce irritation resulting in inflammation and scarring along a wound and thus would be clinically disadvantageous.

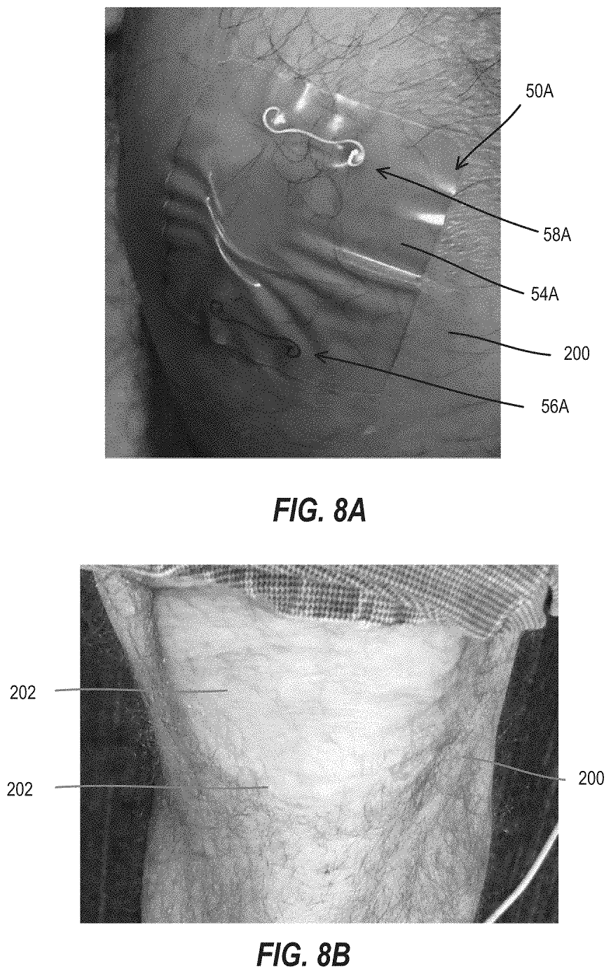

FIG. 8A is a color photograph of an example of wound closure device 50A of the present application applied to tissue 200. Wound closure device 50A comprises backing 54A, micro-structure array 56A and micro-structure array 58A, as described herein. In the illustrated example, tissue 200 comprises tissue at or proximate to a the knee patella. As can be seen, backing 54A can be conformable to curve around the contours of the patella without one of micro-structure arrays 56A and 58A being positioned around a portion of the patella, i.e., micro-structure arrays 56A and 58A can be positioned superiorly and inferiorly of the patella to engage tissue of the skin. The device leads to increased conformability, flexibility, and elasticity over the patella. This results in decreased skin irritation, inflammation, pain and scarring. that can lead to pain and scarring. In contrasts, micro-structures that are located near one another would create a relatively rigid wound closure that would be difficult to conform to the patella making device application challenging and leading to an increased risk of detachment. Such a device would also result in increased irritation, inflammation, and scarring.

FIG. 8B is color photograph of tissue 200 of FIG. 8A after application of tissue closure device 50A of FIG. 8A to tissue 200 for nine days. As can be seen, tissue 200 is relatively free of inflammation, but for the inclusion of minor irritation at locations 202 from micro-structure arrays 56A and 58A.

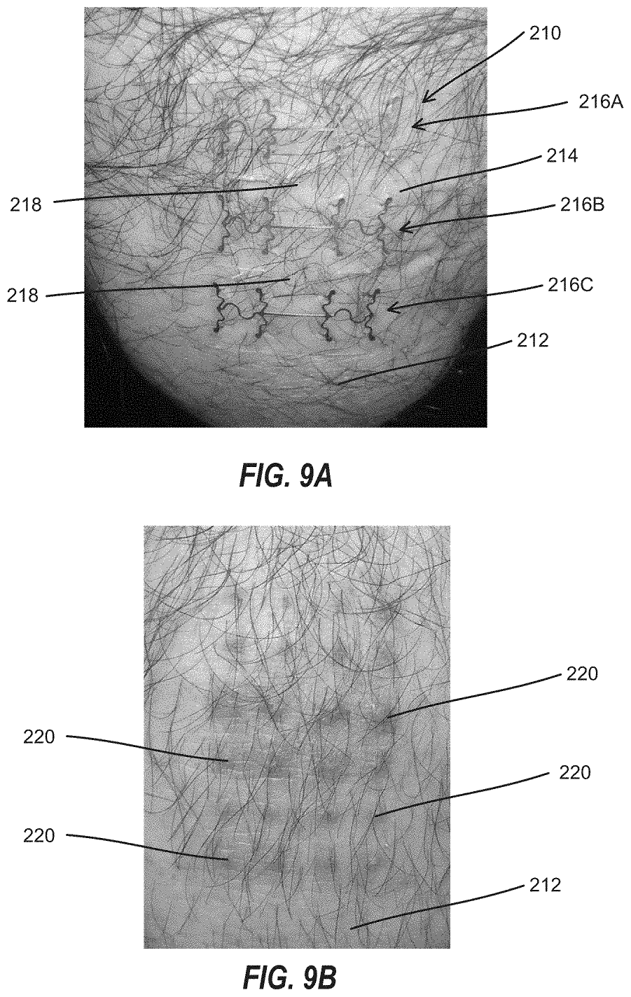

FIG. 9A is a color photograph of a previous wound closure device 210 applied to tissue 212. Wound closure device 210 can comprise backing 214, micro-structure array 216A, micro-structure array 216B, micro-structure array 216C, and slits 218. As can be seen in FIG. 9A, each of micro-structure arrays 216A, 216B and 216C can include four micro-structures at opposite ends of a bridge. As such, each micro-structure array can include eight micro-structures, with device 210 comprising twenty-four micro-structures.

FIG. 9B is a color photograph of the tissue 212 of FIG. 9A after the application of wound closure device 210 for nine days. As can be seen in FIG. 9B, tissue 212 includes twenty-four locations of irritation 220 that correspond to the location s of the micro-structures of micro-structure arrays 216A, 216B and 216C. Thus, device 210 produces a not only a large number of irritation locations due to the large number of micro-structures, but the irritation at each location appears to be exacerbated by the large number. As such, device 50A of FIGS. 8A and 8B not only reduces the number direct irritation locations by reducing the number of micro-structures, but reduces the irritation at each location by reducing the density of micro-structures.

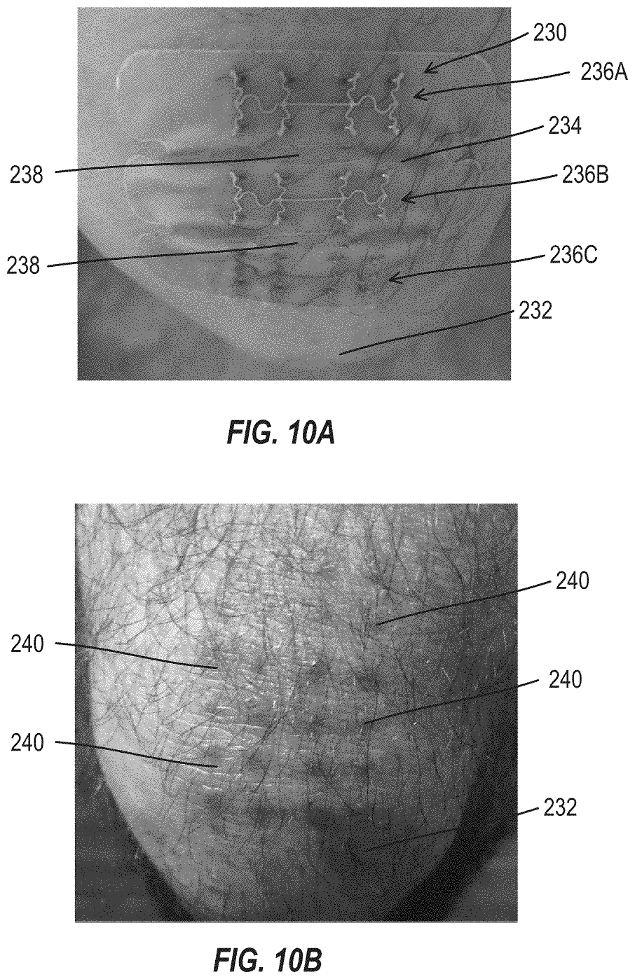

FIG. 10A is a color photograph of a previous wound closure device 230 applied to tissue 232. Wound closure device 230 can comprise backing 234, micro-structure array 236A, micro-structure array 236B, micro-structure array 236C, and slits 238. As can be seen in FIG. 10A, each of micro-structure arrays 236A, 236B and 236C can include four micro-structures at opposite ends of a bridge. As such, each micro-structure array can include eight micro-structures, with device 230 comprising twenty-four micro-structures.

FIG. 10B is a color photograph of tissue 232 of FIG. 10A after application of wound closure device 230 for twelve days. As can be seen in FIG. 10B, tissue 232 includes twenty-four locations of irritation 240 that correspond to the location s of the micro-structures of micro-structure arrays 236A, 236B and 236C, Thus, device 230 produces a not only a large number of irritation locations due to the large number of micro-structures, but the irritation at each location appears to be exacerbated by the large number. As such, device 50A of FIGS. 8A and 8B not only reduces the number direct irritation locations by reducing the number of micro-structures, but reduces the irritation at each location by reducing the density of micro-structures.

With reference to FIGS. 8A-10B, wound closure devices were applied to the skin over the patella. The wound closure devices remained on the skin for 9 or 12 days. Photos of the sites of application were taken immediately after application and upon removal on Days 9 or 12. For Subject 1 (FIGS. 8A and 8B), both the “Elastic Device”, in which the micro-structure arrays are placed at long distances from one another (described in this patent application), and the “Standard Device” (not illustrated) in which the micro-structure arrays are located next to each other were tested. For Subjects 2 (FIGS. 9A and 9B) and 3 (FIGS. 10A and 10B), only the “Standard Device” was tested.

A plastic surgeon rated the degree of inflammation at Days 9 or 12. The rating system was based on the Visual Analog Scale (VAS), which is a sliding scale rating inflammation from none (score of 0) to the worst possible (score of 100). Results are as follows:
Subject 1 (Day 9)
   Elastic Device—score of 1
   Standard Device—score of 30
Subject 2 (Day 9)
   Standard Device—score of 25
   Elastic Device—not tested
Subject 3 (Day 12)
   Standard Device—score of 35
   Elastic Device—not tested

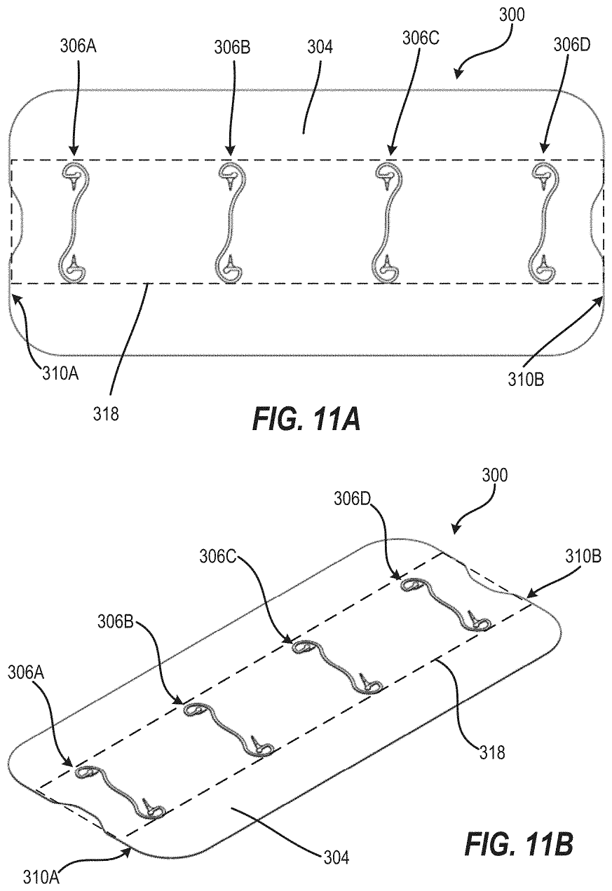

FIG. 11A is a plan view of a wound closure device 300 of the present disclosure incorporating four micro-structure arrays 306A, 306B, 306C and 306D at spaced intervals along backing 304 to produce elongate wound closure spaces between pairs of arrays. FIG. 11B a perspective view of wound closure device 300 of FIG. 11A. FIGS. 11A and 11B are discussed concurrently. Micro-structure arrays 306A-306D can be configured similarly as micro-structure arrays 16A, 16B, 18A, 18B 56A 56B, 58A and 58B, though numbering and discussion is omitted for brevity. Backing 304 can be configured similarly to backings 14A, 14B, 54A ang 54B, but is wider to accommodate more than two micro-structure arrays. In particular, backing 304 can be wide enough to incorporate three or more micro-structure arrays, with elongate wound closure spaces as discussed herein provided between pairs of arrays. In particular, elongate wound closure space 318 can extend across the entire longitudinal width of backing 304 from edge 310A and edge 310B. However, backing 304 does not include a high density of micro-structure arrays to complete elongate wound closure space 318. For example, micro-structure arrays 306A and 306B are spaced at an interval that is similar to that described with reference to FIG. 6 to accomplish uniform or near uniform wound closure between arrays 306A and 306B, micro-structure arrays 306B and 306C are spaced at an interval that is similar to that described with reference to FIG. 6 to accomplish uniform or near uniform wound closure between arrays 306B and 306C, and micro-structure arrays 306C and 306D are spaced at an interval that is similar to that described with reference to FIG. 6 to accomplish uniform or near uniform wound closure between arrays 306C and 306D. Thus, as described herein, uniform or near uniform wound closure strength of device 300 can be achieved from edge 310A to 310B without the need for five or more micro-structure arrays that can impede conformability and such, as described herein.

### NOTES AND EXAMPLES

The following, non-limiting examples, detail certain aspects of the present subject matter to solve the challenges and provide the benefits discussed herein, among others.

In one example, the wound closure device is longer than it is wider.

In another example, the wound closure device is wider than it is longer.

In another example, a wound closure device contains more than two micro-structure arrays in the wound closure device separated by spacing between each of the arrays thus allowing closure of longer wounds using a single device.

In another example, the location of the micro-structure arrays in the device may vary. For example, they may be located immediately adjacent to the ends of the adhesive backing or at an intermediate location on the adhesive backing. For example, they could be located 0.1, 0.2, 0.3, 0.4, 0.5, 0.6, 0.7, 0.8, 0.9, 1, 1.5, or 2 cm from the end of the adhesive backing.

In another example, the micro-structure arrays are of different sizes, including their width and length.

In another example, the number of micro-structure arrays can vary in the device. For example, up to 12 micro-structure arrays can be contained in the device.

In another example the number of micro-structures per array can be varied. For example, they may contain up to 12 micro-structures per array. Different shapes and sizes of micro-structures and micro-structure arrays can be attached to a single adhesive backing in a wound closure device.

In another example, the micro-structure array can be incorporated into the device that is not perpendicular to the edge of the width direction of the device (IS THIS 26A AND 28A OR 26B AND 28B?). For example, it can be at a 10, 20, 30, 40, 50, 60, 70, or 80 degree angle with respect to the width direction of the device.

In another example, the wound closure device can be of varying length. For example, the adhesive backing can be 0.5, 0.6, 0.7, 0.8, 0.9, 1, 1.5, 2, 3, 4, 5, 6, 7, 8, 9, 10, 15, 20, 30, 40, or 50 cm in length.

In another example, the wound closure device can be of varying length. For example, the wound closure device can be 0.1, 0.2, 0.3, 0.4, 0.5, 0.7, 0.8, 0.9, 1, 15, 2, 3, 4, 5, 6, 7, 8, 9, 10, 20, 30, or 50 cm in length.

In another example, the wound closure device can be in different shapes. For example, it can be in rectangular, square, trapezoidal, circular, oval, diamond, pyramid, or another shape.

In another example, the backing can be in different shapes. For example, it can be in rectangular, square, trapezoidal, circular, oval, diamond, pyramid, or another shape.

In another example, the backing can contain apertures. These apertures can be circular, oval shaped, square, rectangular, diamond, or pyramid in shape, The apertures can vary in number.

In another example, the elasticity of the backing can vary.

In another example, the flexibility of the backing can vary.

In another example, the conformability of the backing can vary.

In another example, the wound closure device can be formed into a roll containing more than one micro-structure array.

In another example, the roll may also contain an applicator.

In another example, the surface area between the micro-structure areas represents less than 1%, 2%, 3%, 4%, 5%, 6%, 7%, 8%, 9%, 10%, 20%, 30%, 40% of the total surface area of the wound closure device.

In another example, the surface area of the adhesive backing to which the micro-structure arrays are attached represents more than 95%, 90%, 80%, 70%, or 60% of the total surface area of the wound closure device.

In another example, the length of the micro-structure array can vary. For example, it can be 0.3, 04, 0.5, 1, 1.5, 2, 3, 4, or 5 cm in length.

In another example, the length of the micro-structure array is less than 10%, 20%, 30%, 40%, 50%, 60%, 70%, 80%, 90% of the length of the wound closure device.

In another example, the length of the micro-structure array is less than 10%, 20%, 30%, 40%, 50%, 60%, 70%, 80%, 90%, 100%, 150%, 200%, or 500% of the distance between two micro-structure arrays.

In another example, the width of the micro-structure array can vary. For example, it can be 0.05, 0.1, 0.2, 0.3, 0.4, 0.5, 1, 1.5, 2, 3, 4, or 5 cm in width.

In another example, the width of the microstructure array is less than 1%, 5%, 10%, 20%, or 30% of the distance between the two arrays.

In another example, the distance between the micro-structure arrays can vary. The distance can be greater than 0.1, 0.2, 0.3, 0.4, 0.5, 1, 1.5, 2, 3, 5, 7, or 10 cr.

In another example, the area between two micro-structure arrays is more than 40%, 50%, 60%, 70%, 80%, 90%, or 100% of the area of the adhesive backing to which the microstructure array is attached.

In another example, the adhesive backing that is not attached to the micro-structure array represents more than 70%, 80%, 90%, or 95% of the surface area of the entire adhesive backing of the device.

In another example, the adhesive is only present on a portion of the backing. For example the adhesive could be on 10%, 20%, 30%, 50%, 75%, or 90% of the backing.

In another example, the wound closure device contains apertures that allow drainage of blood and exudate from the wound.

In another example, the wound closure does not contain apertures enabling secure closure of the wound reducing entry of bacteria into the wound.

In another example, the adhesive backing can contain slits or other openings that further increase the flexibility and elasticity of the device further enhancing conformability to the skin surface.

In another example, the wound closure device is applied such that the micro-structure arrays are attached beyond the ends of the wound and not over the wound. Tension between the two micro-structure arrays is sufficient to close the wound even though the micro-structure arrays are not located over the wound. Such an application will provide nearly completely even tension across the wound reducing inflammation and scarring. By placing the arrays outside of the wound, it also reduces further skin irritation that occurs in the wound area. It also eliminates the micro-structures entering the skin in the wound area reducing inflammation and scarring and the risk of infection.

In another example, the micro-structure device can be used to close acute wounds, such as lacerations, port sites, surgical incisions, and skin tears.

In another example, the micro-structure device can be used to close chronic wounds, such as diabetic ulcers, pressure ulcers, venous ulcers, and others. With long distances between the micro-structure arrays, it is possible to apply the device in such a way that it the micro-structures do not enter the wound itself. This would reduce pain, inflammation, tissue injury resulting in poor healing, and risks of infection that would occur if the micro-structures are contained throughout the device in which case they would enter the wound when the device is applied.

In another example, the micro-structure device can contain therapeutic agents. For example, they may contain an antibiotic, hemostatic agent, wound healing, or anti-scarring agent.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more,” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A wound closure device, comprising:
a backing comprising:
a width configured to extend along at least a portion of a wound from a first end to a second end;
a length configured to extend transversely across the wound;
an adhesive layer attached to the backing;
a first micro-structure array attached to the backing proximate the first end, the first micro-structure array configured to extend transversely across the wound; and
a second micro-structure array attached to the backing proximate the second end, the second micro-structure array configured to extend transversely across the wound; and
an elongate wound closure space between the first and second micro-structure arrays.

2. The device of claim 1, wherein the backing comprises an elastic material.

3. The device of claim 2, wherein the backing comprises a silicone sheet.

4. The device of claim 2, wherein the backing comprises a polyurethane sheet or film.

5. The device of claim 1, wherein the backing comprises:
a first indentation located along an edge of the backing at the first end; and
a second indentation located along an edge of the backing at the second end.

6. The device of claim 1, wherein the backing comprises a slit positioned between the first micro-structure array and the second micro-structure array.

7. The device of claim 6, wherein the slit is oriented in a direction substantially parallel with the length of the backing.

8. The device of claim 1, wherein the first micro-structure array and the second micro-structure array each comprise first and second micro-structures.

9. The device of claim 8, wherein the first and second micro-structures are located at opposite axial ends of the first and second micro-structure arrays.

10. The device of claim 8, wherein the first micro-structure array and the second micro-structure array collectively define four micro-structures arranged in a pattern defining a rectilinear shape.

11. The device of claim 1 wherein each of the first micro-structure array and the second micro-structure array comprises:
a non-stretchable bridge extending in a longitudinal direction;
a first spring structure positioned at a first end of the bridge;
a second spring structure positioned at a second end of the bridge;
a first micro-structure connected to the first spring structure; and
a second micro-structure connected to the second spring structure.

12. The device of claim 11, wherein the first micro-structure and the second micro-structure are aligned along a central axis of the bridge.

13. The device of claim 1, wherein the first micro-structure array and the second micro-structure array each comprise a bridge extending along a central axis configured to extend across a wound.

14. The device of claim 13, wherein the central axis of each bridge is positioned at or within a first distance from a transverse edge of the backing configured to extend transverse to the wound.

15. The device of claim 14, wherein the first micro-structure array and the second micro-structure array are longitudinally separated by a second distance, the second distance being greater than the first distance.

16. The device of claim 15, wherein the backing does not include a micro-structure array along the second distance.

17. The device of claim 15, wherein the first distance is within 25% of a total width of the backing.

18. The device of claim 17, wherein the second distance is approximately 50% of the total width of the backing.

19. The device of claim 15, wherein the second distance is approximately 66% of the total width of the backing.

20. The device of claim 19, wherein the second distance is approximately 80% of the total width of the backing.

21. The device of claim 19, wherein the second distance is approximately 90% of the total width of the backing.

22. The device of claim 19, wherein the second distance is approximately 95% of the total width of the backing.

23. The device of claim 1, wherein the first micro-structure array and the second micro-structure array are each configured to provide longitudinal closing strength longitudinally from each of the first and second micro-structure arrays.

24. The deice of claim 20, wherein the first micro-structure array and the second micro-structure array are each configured to provide approximately uniform closing strength longitudinally across the backing.

25. The device of claim 20, wherein the longitudinal closing strength of the first micro-structure array and the second micro-structure array overlap in a longitudinal direction.

26. The device of claim 23, wherein peak transverse closing strength of the first micro-structure array does not overlap with peak transverse closing strength of the second micro-structure array.
